(19) United States
(12) Patent Application Publication
(10) Pub. No.: US 2023/0268078 A1
(43) Pub. Date: Aug. 24, 2023

(54) METHOD AND SYSTEM FOR GENERATING A VISUAL REPRESENTATION

(71) Applicants: Oxford University Innovation Limited, Oxford (GB); GROUND TRUTH LABS LTD, Oxford (Oxfordshire) (GB)

(72) Inventors: Alan ABERDEEN, Oxford (Oxfordshire) (GB); Daniel ROYSTON, Oxford (Oxfordshire) (GB); Helen THEISSEN, Oxford (Oxfordshire) (GB); Korsuk SIRINUKUNWATTANA, Oxford (Oxfordshire) (GB); Jens RITTSCHER, Oxford (Oxfordshire) (GB)

(21) Appl. No.: 18/012,675

(22) PCT Filed: Jun. 28, 2021
(86) PCT No.: PCT/GB2021/051630

§ 371 Date: Dec. 23, 2022

(30) Foreign Application Priority Data
Jun. 26, 2020 (GB) 2009762.2

(51) Int. Cl. G16H 50/50; G16H 50/20; G06V 20/69

(52) U.S. Cl. CPC G16H 50/50; G16H 50/20; G06V 20/698

(57) ABSTRACT

Methods and systems for generating visual representations of variation of disease-relevant classification are disclosed. Training data is received that comprises sample data units from subjects that represent information about a biological sample via an N-dimensional set of values. A dimensionality reduction algorithm represents each sample data unit as a respective point in a reduced dimension parameter space. Distributions of points from the dimensionality reduction are used to derive a probability density distribution for each of a plurality of disease-relevant classifications in the reduced dimension parameter space. A visual representation of each of the derived probability density distributions in the reduced dimension parameter space is generated to provide a visual representation of disease-relevant classification variation over the parameter space.

The present disclosure relates to computer-implemented methods and systems for generating a visual representation of variation of a disease-relevant classification over a parameter space representing biological samples from human and/or animal subjects. The visual representation may be generated from morphological and/or topological characteristics of objects such as cells in biological samples, for example to display information supporting diagnosis of Philadelphia-negative myeloproliferative neoplasms (Ph-MPN).

Ph-MPN are a group of disorders in which acquired mutations in haematopoietic stem cells affecting the MPL-JAK-STAT signalling pathway drive excessive proliferation of one or more blood lineages. The three most common Ph-MPN (essential thrombocythaemia [ET], polycythaemia vera [PV] and myelofibrosis [MF]) have overlapping clinical and laboratory features that can make their distinction challenging, particularly at early disease time points. In >90% of MPN, mutations are detected in one of three genes: JAK2, CALR and MPL. Mutations in JAK2, typically JAK2V617F, are detected in almost all cases of PV and >60% of ET and MF, while mutations in CALR and MPL occur in ET and MF. Around 5-10% of patients with ET and MF do not have a detectable ‘driver’ mutation and can be difficult to distinguish from ‘reactive’ causes.

Currently, subjective histological features form an important component of the classification scheme central to the diagnosis of MPN, and are integral to the diagnosis of other human cancers. Recent advances in computational image analysis have the potential to transform the conventional morphological assessment of human tissues. In particular, quantification of specific cell populations and systematic description of tissue architecture can replace or augment the categorical classification systems that are currently employed in the diagnosis of cancer. Moreover, translation of advanced tissue and single-cell-based genomic and proteomic technologies into clinical strategies will require sophisticated approaches to the assessment of complex pathological tissues that are beyond the scope of routine histopathology.

In MPN, accurate classification is crucial for optimal management as treatment targets and risks of progression differ between the disorders. In ET and MF cases that lack a mutation in one of the three main MPN driver genes (so-called triple negative [TN]), their distinction from a ‘reactive’ process, for example due to chronic inflammation, remains particularly challenging. This is reflected in the revised 2016 WHO Classification scheme of myeloid malignancies, in which particular emphasis is placed upon the integration of clinical, genetic and histological features for the diagnosis of MPN. Central to the histological interpretation of bone marrow trephines (BMTs) from suspected MPN patients is the assessment of megakaryocytes using long-established but highly subjective descriptions of their cytological and topographic features. These include variations in cell size, atypia (nuclear lobulation/complexity etc.), and cell clustering which may be subconsciously based on assessment of only a subset of the megakaryocytes examined in the tissue section. Despite their importance in the 2016 WHO classification system, there is controversy about the relative significance and reliability of these subjective cytological descriptions.

It is an object of the invention to enable relevant information to be extracted more easily and/or reliably from biological samples taken from human subjects.

According to an aspect of the invention, there is provided a computer-implemented method of generating a visual representation of variation of a disease-relevant classification over a parameter space representing biological samples from human and/or animal subjects, the method comprising: receiving training data comprising, for each of a plurality of human and/or animal subjects, at least one sample data unit comprising information about a biological sample taken from the subject, the training data also comprising a disease-relevant classification of the subject when the biological sample was taken, the information about the biological sample being represented in each sample data unit by an N-dimensional set of values, where N>2; using a dimensionality reduction algorithm to represent each sample data unit as a respective point in a reduced dimension parameter space; processing the resulting distributions of points for each of a plurality of disease-relevant classifications to derive a probability density distribution for each of the disease-relevant classifications in the reduced dimension parameter space; and generating a visual representation of each of the derived probability density distributions in the reduced dimension parameter space, thereby providing a visual representation of disease-relevant classification variation over the parameter space.

The reduced dimension parameter space may for example be a two-dimensional parameter space or a three-dimensional parameter space. Two-dimensional and three-dimensional parameter spaces can be visualised easily on a two-dimensional display screen.

The computer-implemented generation of the visual representations of probability density distributions provides a tool for users (e.g. clinicians) that enables a more informative comparison to be made between different sample data units. In particular, a more in-depth and/or reliable comparison can be made between a new (previously unseen) sample data unit and a library of sample data units obtained in the past and associated with a range of disease-relevant classifications. The tool facilitates more efficient comparison at diagnosis time between new patient samples and library samples of normal and disease tissue. This can either be an initial diagnostic assessment without immediate expert pathology review, or may provide immediate critical feedback to pathologists who wish to establish the likelihood that a proposed diagnosis is correct and/or whether further assessment/investigation should be considered.

The visualisation in the reduced dimension parameter space (e.g. in 2D or 3D) allows changes in patient samples resulting from established or novel therapies to be grouped together and assessed by treatment regimen rather than just disease. In other words, samples that may look very different such as indolent ET and fibrotic PV may change in a similar or related way when viewed in the reduced dimension parameter space. Such changes would be difficult or impossible to identify using higher dimensional representations.

It is furthermore possible to map from each point in the reduced dimension parameter space back to the corresponding point in the N-dimensional space of the original sample data units. This possibility of transitioning between the reduced and N-dimensional representations allows differences or changes between samples (e.g. morphological differences/changes) to be understood and interrogated in more detail. The N-dimensional representation may, for example, be such as to allow prediction of disease relevant endpoints, such as histopathology grades or response to therapy. For example, the mapping into the original N-dimensional space could be used to identify a sub-populations of patients. The tool for generating the visual representation in the reduced dimension parameter space thus facilitates effective interpretation of points in the original N-dimensional space.

The methodology has been exemplified in the context of analysing bone marrow features. The statistical approach taken is a significant departure from conventional histological descriptions in this area. In the example described, automated identification and quantification of nine distinct megakaryocyte cell subtypes facilitates an unbiased, accurate summary and description of the entire megakaryocyte population within a given BMT sample. The approach is combined with consideration of topographical data summarising the distribution of each cell subtype throughout the marrow space. The generation of the visual representations makes it possible for a user to visualize complex megakaryocyte morphological features in a manner readily appreciable by non-expert pathologists, haematologists inexperienced in trephine reporting, and their patients.

The described approach allows detailed comparisons to be made between specific patient cohorts, such as annotated trial cohorts, patients with different mutation status (including key MPN driver mutations), and other common laboratory or clinical features. In an example implementation, eight megakaryocyte subtypes were found to be significantly associated with an MPN diagnosis. While some of these phenotypic groups, e.g. large atypical cells forming clusters, are key pathological hallmarks of conventional classification schemes, new and more subtle morphological features were identified using the described platform that would not have been recognised by conventional approaches.

The described approach may also be used to compare between species. This may be of particular value for example where transgenic mouse models of disease are used to evaluate the influence of genetic, epigenetic disease modifiers etc. or the effect of novel therapies.

In some embodiments, the method further comprises: receiving a new sample data unit from a subject to be assessed; and calculating the position of a point in the reduced dimension parameter space representing the received new sample data unit. In an embodiment, a plurality of the new sample data units are received for the same subject at different times and the positions of plural respective points are calculated and represented visually in registration with the generated visual representation of the derived probability density distributions. Alternatively or additionally, a plurality of the new sample data units are received for different subjects and the positions of plural respective points are calculated and represented visually in registration with the generated visual representation of the derived probability density distributions.

The ability to review an index case (a new sample data unit) in the context of a larger annotated tissue cohort is of particular value, as it allows the interpretation of megakaryocyte populations in the context of large libraries of reactive and MPN samples. Further, it is possible to visualise changes in the megakaryocyte population in serial sections over the course of disease progression. Analysis of serial patient samples allows changes in tissue morphology to be accurately detected and quantified, particularly with respect to disease progression or tissue normalisation. This approach is exemplified herein by the inventors, who demonstrate clear evidence of a shift in the location of points representing sample data units on a PCA plot (including an example of the visual representations of probability density distributions) from two patients who developed MF-like appearances following progression of ET and PV. This demonstrates the potential of the platform in the routine monitoring and assessment of patients receiving conventional or novel therapeutic interventions, particularly where specialist haematopathologist expertise is not available. Of note there is clear overlap of various regions describing the reactive and MPN sample subgroups on the PCA plot, consistent with the diagnostic ambiguity of a significant proportion of MPN patients when classified using WHO criteria.

In the example described, the approach provides a comprehensive and easily interpreted summary of the megakaryocytic population that will enable the pathologist to concentrate on the ‘higher-level’ process of integrating the broader pathological features with the clinical and laboratory findings. The approach is ideally suited for more accurate assessment of sequential specimens from patients undergoing treatment and/or repeated investigation, in whom quantitative morphological correlates of disease response are currently unavailable. In some arrangements, the method further comprises performing a cluster analysis to identify a plurality of clusters of points representing the sample data units. The cluster analysis may identify regions in the reduced dimension parameter space and/or N-dimensional space where sample data units have common features, separately from the disease-relevant classifications. The points representing the sample data units (on which the cluster analysis is performed) may comprise points (or a subset of points) in the reduced dimension parameter space or points (or a subset of points) in the original N-dimensional space.

In some arrangements, the generated visual representation comprises cluster-boundary indicators representing locations of the identified clusters. The generated visual representation may further comprise a higher-dimensional sample representation for each of one or more representative sample data units, each higher-dimensional sample representation having more dimensions than the reduced dimension parameter space (e.g. three or more dimensions in the case where the reduced dimension parameter space is a 2D space). The inclusion of such higher-dimensional sample representations, which may be represented by radar plots for example, makes it easier for a user to recognise relationships between different regions in the reduced dimension parameter space and corresponding changes in recognizable features of the sample data units, such as differences in cell morphologies or topological distributions of the cells.

In some arrangements, each higher-dimensional sample representation is visually associated with a respective one of the identified clusters and the representative sample data unit represents one or more sample data units located in the cluster, optionally representing an average over sample data units located in the cluster. Associating higher-dimensional sample representations with sample data units in clusters and/or averages over sample data units in clusters maximises the useful information content of the displayed higher-dimensional sample representations. This makes it easier, for example, for a user to relate differences in position in the reduced dimension parameter space associated with patient trajectories over time, or different patients, to corresponding observable differences in patient data associated with sample data units, such as observable differences in cell morphologies or topological distributions of the cells.

According to an alternative aspect of the invention, there is provided a system for generating a visual representation of variation of a disease-relevant classification over a parameter space representing biological samples from human and/or animal subjects, comprising: an imaging device configured to capture an image of a biological sample; and a data processing system configured to process a sample data unit comprising information derived from the image of the biological sample, wherein the processing comprises: obtaining an N-dimensional set of values representing morphological and/or topological characteristics of objects in the image of the biological sample; using a dimensionality reduction algorithm to represent each sample data unit as a respective point in a reduced dimension parameter space having fewer than N dimensions; and generating a visual representation of the sample data unit in the reduced dimension parameter space together with a visual representation of probability density distributions for each of plural disease-relevant classifications in the reduced dimension parameter space.

Embodiments of the disclosure will be further described by way of example only with reference to the accompanying drawings, in which:

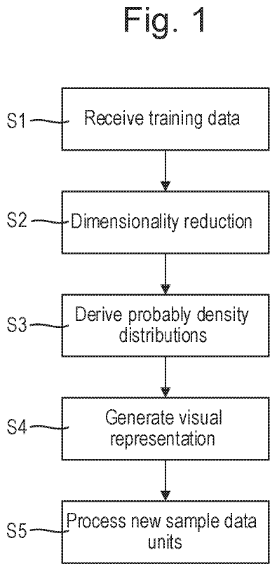

FIG. 1 is a flow chart depicting a framework for an example method of generating a visual representation of variation of a disease-relevant classification over a parameter space representing biological samples from human and/or animal subjects;

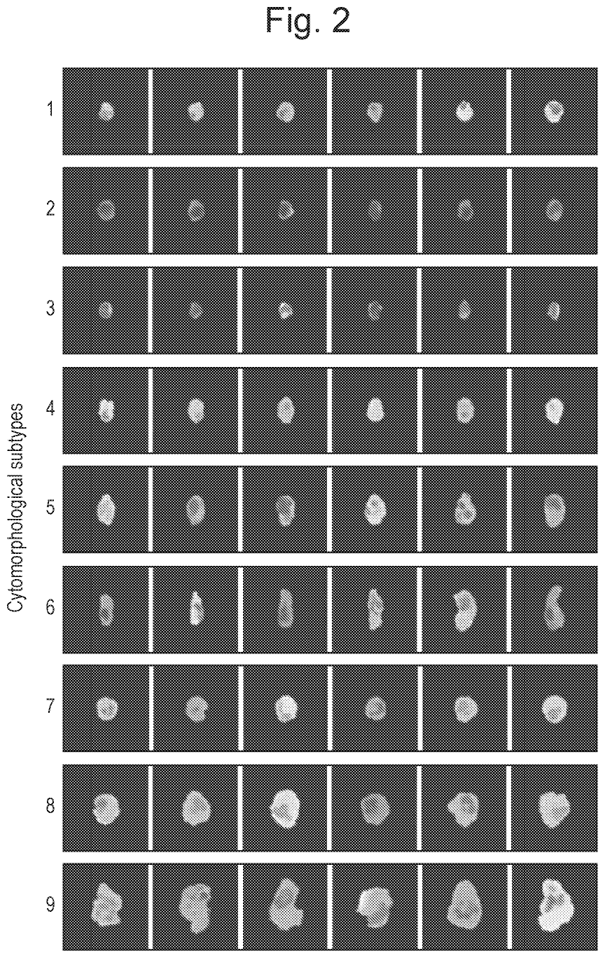

FIG. 2 depicts assignment of five example cells (columns) to each of nine different megakaryocytic subtypes (rows) automatically discovered in an unsupervised clustering analysis;

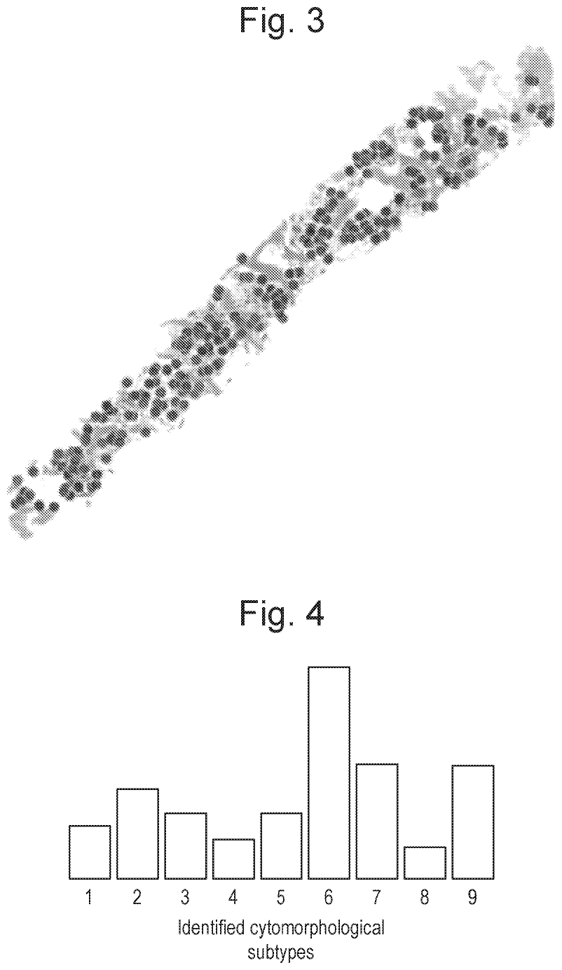

FIG. 3 shows an image of a sample with locations of different cytomorphological subtypes shown by circles having respective different shades;

FIG. 4 is a histogram showing a distribution of cells in the sample of FIG. 3 between the nine megakaryocytic subtypes of FIG. 2;

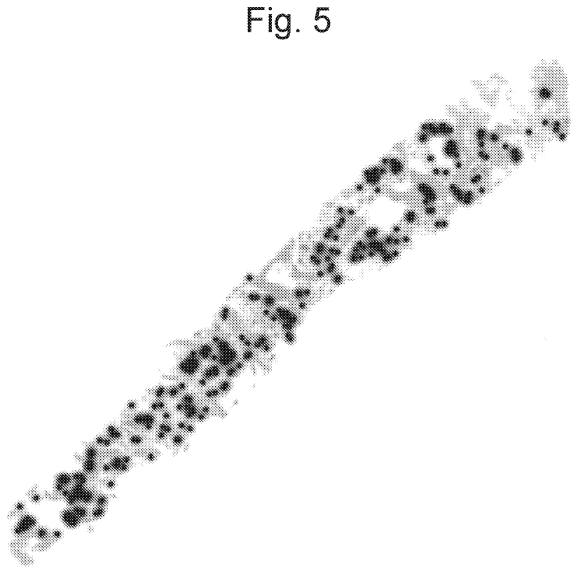

FIG. 5 shows a spatial distribution of cells in the sample of FIG. 3;

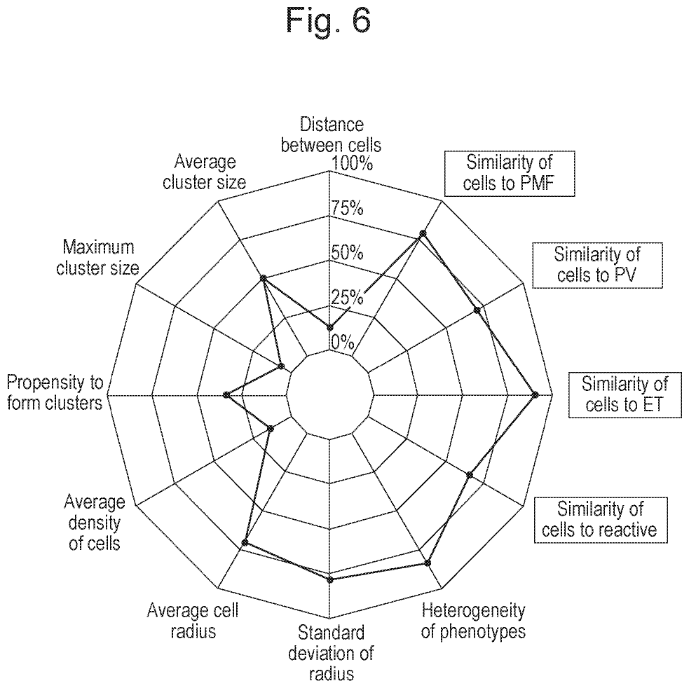

FIG. 6 is a radar plot showing example values of an example N-dimensional set of values representing information about a biological sample;

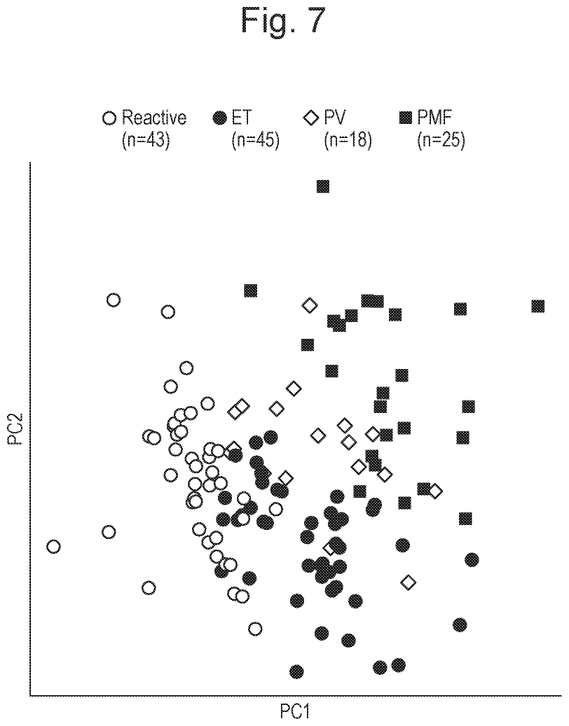

FIG. 7 is a two-dimension plot showing representations of sample data units as points after application of a dimensionality reduction algorithm (principle component analysis);

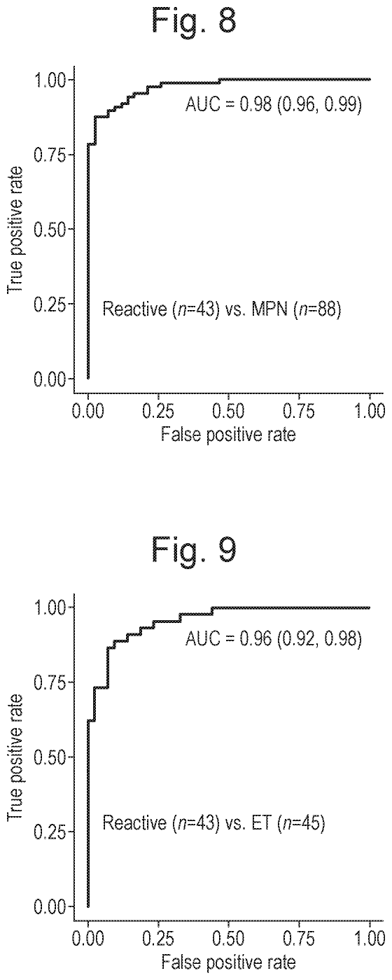

FIG. 8 is a plot showing how a random forest classifier reached an AUC of 0.98 for discriminating reactive and MPN samples using the data from FIG. 7;

FIG. 9 is a plot showing how a random forest classifier reached an AUC of 0.96 for discriminating reactive and ET samples using the data from FIG. 7;

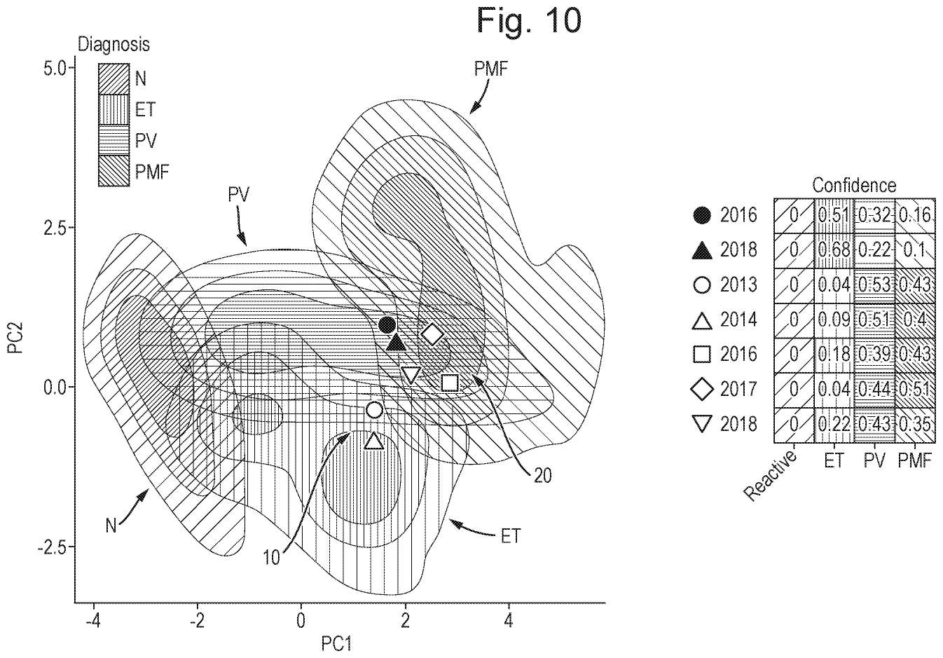

FIG. 10 depicts a visual representation of probability density distributions for four disease-relevant classifications, with plotted points representing sample data units from first and second subjects taken at different times;

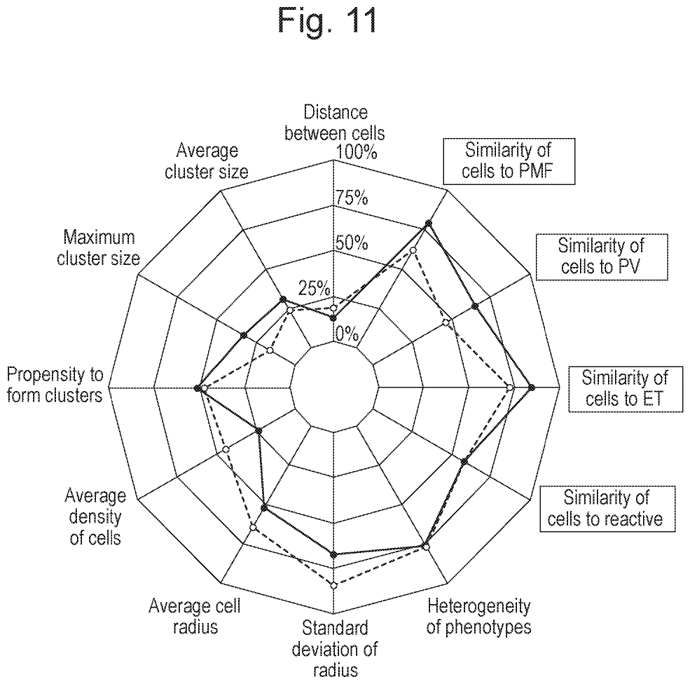

FIG. 11 is a radar plot showing feature values for sample data units from the first subject considered in FIG. 10 taken at different times;

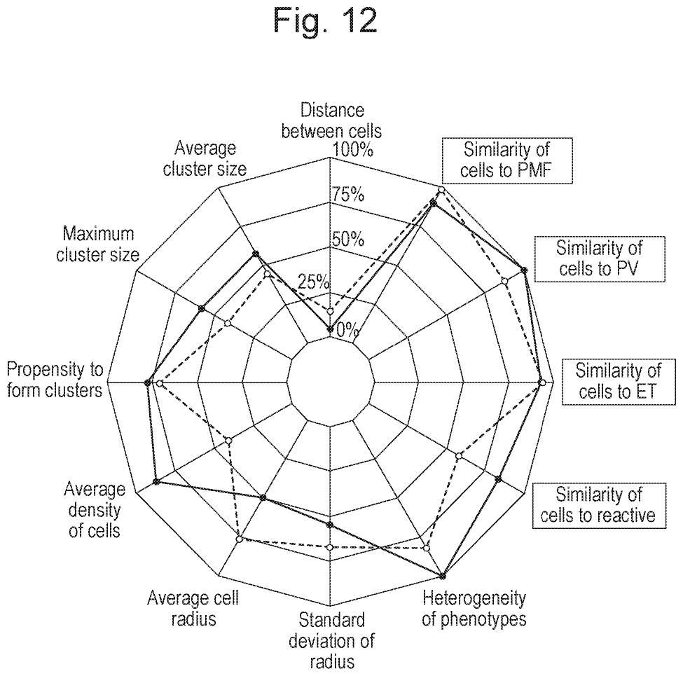

FIG. 12 is a radar plot showing feature values for sample data units from the second subject considered in FIG. 10 taken at different times;

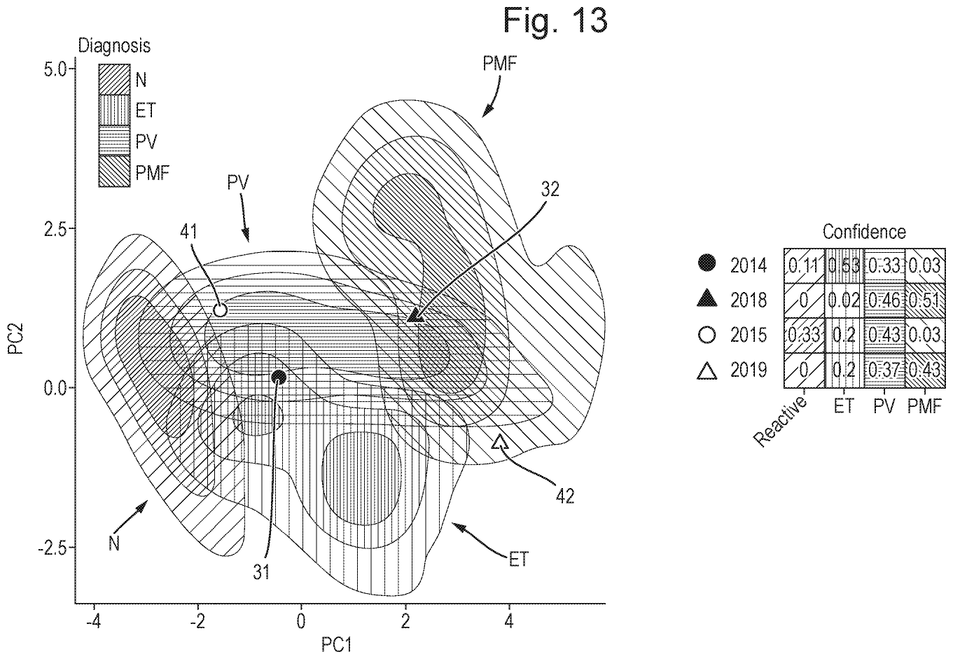

FIG. 13 depicts a visual representation of probability density distributions for four disease-relevant classifications, with plotted points representing sample data units from third and fourth subjects taken at different times;

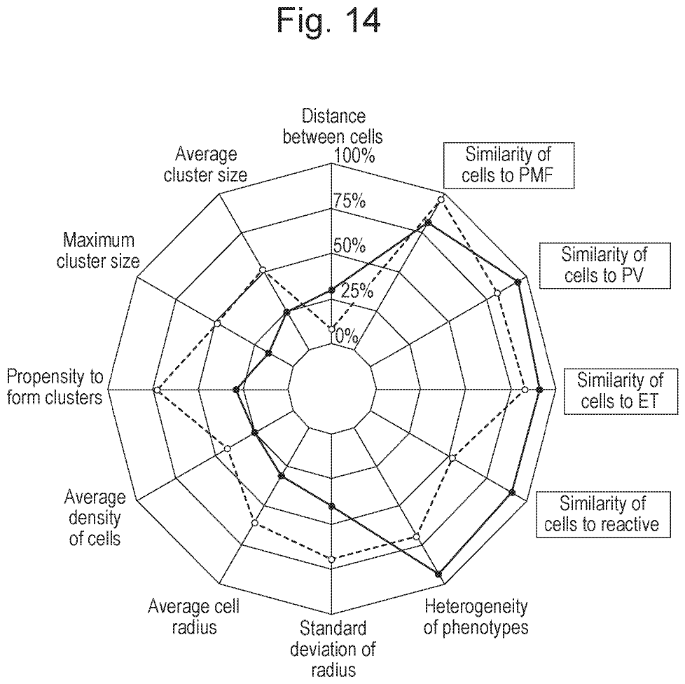

FIG. 14 is a radar plot showing feature values for sample data units from the third subject considered in FIG. 13 taken at different times;

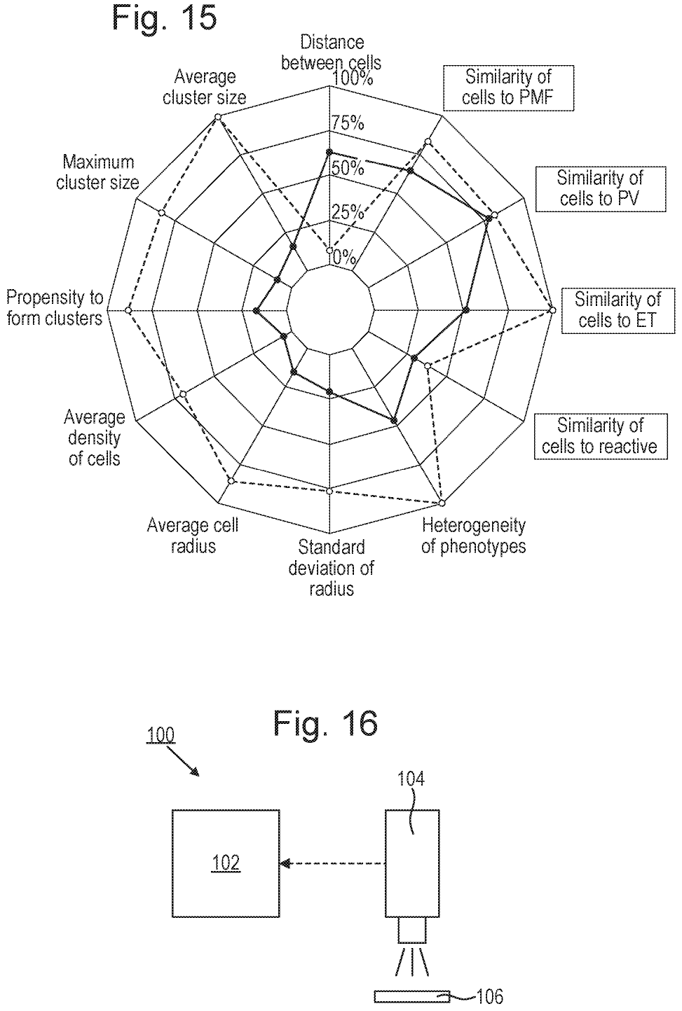

FIG. 15 is a radar plot showing feature values for sample data units from the fourth subject considered in FIG. 13 taken at different times;

FIG. 16 depicts a system for generating a visual representation; and

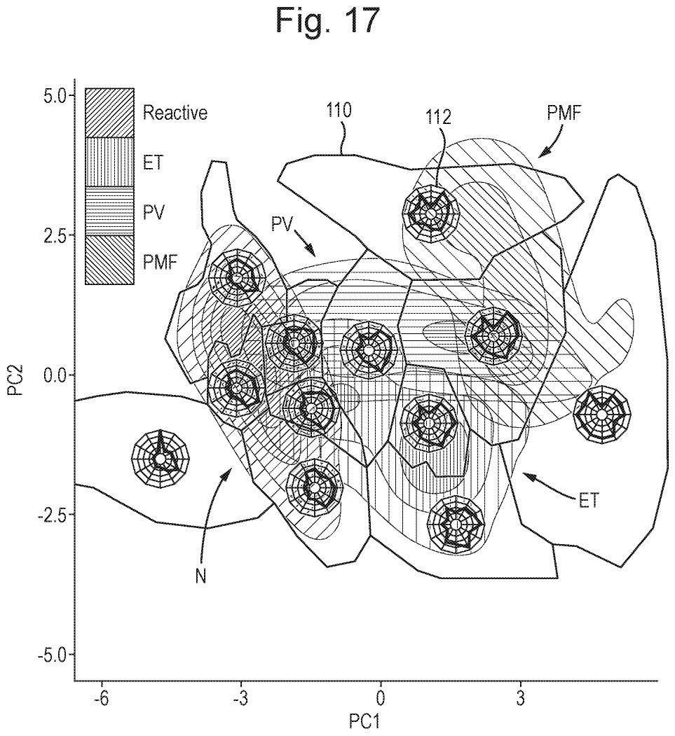

FIG. 17 depicts a visual representation of probability density distributions for four disease-relevant classifications, with superimposed cluster-boundary indicators and higher-dimensional sample representations (radar plots) associated with the clusters.

Embodiments of the disclosure relate to computer-implemented methods of generating a visual representation. Methods of the present disclosure are thus computer-implemented. Each step of the disclosed methods may be performed by a computer in the most general sense of the term, meaning any device capable of performing the data processing steps of the method, including dedicated digital circuits. The computer may comprise various combinations of known computer elements, including for example CPUs, RAM, SSDs, motherboards, network connections, firmware, software, and/or other elements known in the art that allow the computer to perform the required computing operations. The required computing operations may be defined by one or more computer programs. The one or more computer programs may be provided in the form of media or data carriers, optionally non-transitory media, storing computer readable instructions. When the computer readable instructions are read by the computer, the computer performs the required method steps. The computer may consist of a self-contained unit, such as a general-purpose desktop computer, laptop, tablet, mobile telephone, or other smart device. Alternatively, the computer may consist of a distributed computing system having plural different computers connected to each other via a network such as the internet or an intranet.

FIG. 1 is a flow chart depicting a framework for methods of generating a visual representation according to the disclosure. As will be explained below, the visual representation represents a variation of a disease-relevant classification over a parameter space. The parameter space represents biological samples from human and/or animal subjects. The parameters of the parameter space are thus suitable for describing characteristics of the biological samples that are correlated with disease-relevant classifications of interest.

In step S1, the method comprises receiving training data. The training data comprises, for each of a plurality of human and/or animal subjects, at least one sample data unit. Each sample data unit comprises information about a biological sample taken from the respective subject. The training data also comprising a disease-relevant classification of the subject when the biological sample was taken. The information about the biological sample is represented in each sample data unit by an N-dimensional set of values, where N>2.

In some embodiments, the biological sample comprises a stained tissue section taken from the subject. The information about the biological sample may be derived from an image of the section or from a processed representation of the image.

In some embodiments, at least a subset of the dimensions of the N-dimensional set of values comprises parameters representing morphological characteristics of objects in an image of the biological sample. The objects may comprise human and/or animal cells. In an embodiment, as exemplified below, at least a subset of the cells may be megakaryocytes. In embodiments of this type, the disease-relevant classifications may include a classification associated with at least one myeloproliferative neoplasm. The disease-relevant classifications may include a classification associated with each of two or more Philadelphia-negative myeloproliferative neoplasms (Ph-MPN), preferably including one or more of thrombocythaemia (ET), polycythaemia vera (PV), and myelofibrosis (MF). The disease-relevant classifications may additionally include reactive mimics.

In some embodiments, the objects of interest are detected automatically. A machine learning (e.g. deep-learning) approach may be used to detect the objects. The machine learning approach may be trained to generated bounding boxes around candidate objects and calculate a score associated with the presence of an object of interest within the boundary box. An image segmentation algorithm can then be applied within each boundary box to identify a boundary of the object in the boundary box. The cell area is segmented from the background microenvironment.

In some embodiments, objects identified from the training data form a library of object morphologies. Where the objects are cells, the object morphologies may be referred to as cytomorphologies. In an embodiment, the library of objects is analysed to identify a set of discrete morphological subtypes (e.g. cytomorphological subtypes). The identified sub-types may be used to define at least a subset of the dimensions of the N-dimensional set of values. Thus, some of the dimensions may represent information about how objects present in a biological sample are distributed between the identified morphological sub-types.

In some embodiments, the morphological subtypes are identified by performing feature extraction. The feature extraction may thus define at least a subset of the dimensions of the N-dimensional set of values. The feature extraction may be performed using machine learning. In an embodiment, as exemplified below in the detailed example, a neural network (autoencoder) is used. The autoencoder is trained in an unsupervised manner to learn efficient data encodings. A clustering analysis can then be performed to group morphologically similar objects. In the detailed example described below, this approach was used to generate a 10-by-10 grid with 100 megakaryocyte subtypes. Markov clustering was then used to further reduce the number of subtypes to nine distinct cytomorphological subtypes to which each identified megakaryocyte can be assigned, as depicted in FIG. 2.

In some embodiments, at least a subset of the dimensions of the N-dimensional set of values comprises parameters representing a topological distribution of objects within the biological sample. In the example described, a topological distribution of the identified cytomorphological subtypes in the marrow space is determined. An example topological distribution is shown in FIGS. 3-5. FIG. 3 shows an image of a sample with locations of different cytomorphological subtypes shown by circles having respective different shades. FIG. 4 is a histogram showing the distribution of the cells between the nine possible morphological subtypes (the height of each bar representing a proportion of the cells that belong to the respective subtype). FIG. 5 shows an overall spatial distribution of the cells through the marrow space. Information about the topological distribution of objects can be represented by a set of numerical values using machine learning methods (each corresponding to one dimension of the N-dimensional set of values representing the information about the biological sample).

FIG. 6 is a radar plot showing graphically example values of an example N-dimensional set of values representing information about a biological sample. Each radius of the radar plot represents one or more of the N dimensions. The nine cytomorphological subtypes respectively provide nine of the feature values (dimensions). These are represented and visualized in FIG. 6 in the form of four values representing morphological similarity to different subgroups of MPN or reactive cases (each highlighted by a bounding box on the right of the figure in FIG. 6). The remaining eight dimensions are heterogeneity of phenotypes (morphological subtypes) as determined by Shannon's entropy measure (“Heterogeneity of phenotypes”), average and standard deviation of cell radii (“Average cell radius” and “Standard deviation of cell radius”), average spatial density of cells (“Density of cells”), proportion of cells in clusters (“Propensity to form clusters”), maximum number of cells in clusters (“Maximum cluster size”), average number of megakaryocytes in clusters (“Average cluster size”), and the 1st quantile of the distribution of the nearest neighbour distance between megakaryocytes (“Distance between cells”). Generation of radar plots such as that shown in FIG. 6 enable sample data units to be readily compared visually.

Referring again to FIG. 1, in step S2 of the method a dimensionality reduction algorithm is used to represent each sample data unit as a respective point in a reduced dimension parameter space. Thus, the N-dimensional representation of each sample data unit is reduced to a representation having fewer than N dimensions, preferably a two-dimensional representation or a three-dimensional representation. In some embodiments, the dimensionality reduction algorithm using principle component analysis (PCA).

The dimensionality reduction enhances visualisation of multiple sample data units in a cohort by allowing them to be compared within a single displayable plot in an abstract reduced dimension space (learnt by PCA). The sample data units may for example be compared within a single two-dimensional plot in an abstract two-dimensional space (learnt by PCA). An example of such a two-dimensional plot is shown in FIG. 7 for the example described. Four sets of points are shown, each set of points representing sample data units corresponding to a different respective group of subjects (43 reactive, 45 ET, 18 PV and 25 MF). In this example, the PCA shows clear separation of reactive and MPN samples, as well as separation of MPN subtypes. Based on these feature representations, the inventors trained a random forest classifier to distinguish 1) reactive (n=43) and MPN (n=88) samples, and 2) reactive (n=43) and ET (n=45) samples. In five-fold cross validation (used to estimate the performance of a model by which data are split into k groups of approximately equal size) the classifier reached the AUC of 0.98 for discriminating the reactive and MPN samples, and the AUC of 0.96 for discriminating the reactive and ET samples (FIGS. 8 and 9). Step S2 thus produces a plurality of distributions of points in a reduced dimension learnt PCA space (e.g. a two-dimensional learnt PCA space or a three-dimensional learnt PCA space). Each distribution of points is made up of points representing sample data units that are associated with the same disease-relevant classification. In the example described, the disease-relevant classifications include reactive, ET, PV, and MF, but other disease-relevant classifications could be included additionally or as alternatives.

In step S3, each of the distributions of points derived in step S2 (each corresponding to a respective disease-relevant classification) are processed to derive a probability density distribution for each disease-relevant classification in the reduced dimension parameter space. The processing to derive the probability density distributions may be performed, for example, using kernel density estimation. The derived probability density distributions may be used to calculate a confidence score representing a confidence that a sample data unit belongs to a particular one of the disease-relevant classifications. The confidence score may be derived, for example, through a Gaussian kernel density estimation conditional on each subtype in the (e.g. two-dimensional or three-dimensional) PCA space, where P(x|classification) denotes a Gaussian kernel density estimate of a sample x given a disease-relevant classification. In the case of the example described, where the disease-relevant classifications include reactive, ET, PV and MF, the confidence score of a sample belonging to a particular classification is given by:

P(classification|x)=P(x|classification)/[P(x|reactive)+P(x|ET)+P(x|PV)+P(x|MF)].

In step S4, a visual representation of each of the derived probability density distributions in the reduced dimension parameter space is derived to providing a visual representation of disease-relevant classification variation over the parameter space. The generated visual representation may be displayed on a display device or output as data suitable for causing a display to display the generated visual representation. Examples of generated visual representations are shown in FIGS. 10 and 13. In some embodiments, as exemplified in FIGS. 10 and 13, the generated visual representation of the derived probability density distributions comprises plots representing one or more contours of equal confidence. In the examples of FIGS. 10 and 13, four probability density distributions are depicted, respectively for N, ET, PV and MF. Each probability density distribution is depicted as regions of different depths of shading. A darkest central region represents where the probability density is above a first threshold. A medium intermediate shading represents where the probability density is between the first threshold and a second threshold that is lower than the first threshold. A lightest shading represents where the probability density is between the second threshold and a third threshold that is lower than the second threshold. Thus, the darker the shading the higher the probability that a point in that region belongs to the respective disease-relevant classification. Boundaries between regions of different shading for a particular disease-classification represent contours of equal confidence with respect to a particular disease-classification (i.e. equal values of P(x|classification)).

In the case where the reduced dimension parameter space is a three-dimensional parameter space, the visual representation may be more complex than the two-dimensional case but may still be readily achieved. For example, a plot of points in the three-dimensional parameter space may be presented in a perspective view that can be rotated on the screen and/or magnified/demagnified to inspect different parts of the visualisation. Contours of equal confidence can be depicted as surfaces of three-dimensional features, which may optionally be configured to appear partially transparent.

In step S5, the method further comprises receiving a new sample data unit from a subject to be assessed and calculating the position of a point in the reduced dimension parameter space representing the received new sample data unit.

In some embodiments, the calculation of the position comprises using the dimensionality reduction algorithm (e.g. PCA) to reduce the dimensions of the new sample data unit (e.g. to two dimensions or three dimensions).

In some embodiments, the method further comprises determining a disease-relevant classification of the new sample data unit. The determined disease-relevant classification may be derived, for example, by plotting a point representing the new sample data unit at the calculated position in the reduced dimension parameter space and comparing the position to the visual representations of the derived probability density distributions for the different disease-relevant classifications. If the point lies in a darkly shaded region of one of the probability density distributions of one of the disease-relevant classifications and is far from a darkly shaded region of the probability density distribution of any other disease-relevant classification, it may be concluded with relatively high confidence that the new sample data unit has been taken from a subject corresponding to the disease-relevant classification corresponding to the darkly shaded region in which the point is located. In some cases, the location of the point corresponding to the new sample data unit may, however, not correspond as clearly to a single disease-relevant classification. In some embodiments, this situation is allowed for by providing a quantitative measure of confidence in a determined classification. The quantitative measure of confidence is determined using the derived probability density distributions and the calculated position of the point in the reduced dimension parameter space representing the received new sample data unit. The measure of confidence may comprise the confidence score discussed above, derived using the expression P(classification|x)=P(x|classification)/[P(x|reactive)+P(x|ET)+P(x|PV)+P(x|PMF)], where P(x|reactive), P(x|ET), P(x|PV), and P(x|PMF) are derived based on the position of the point relative to each of the four respective probability density distributions.

In some embodiments, as exemplified in FIGS. 10-13, a plurality of the new sample data units are received for the same subject at different times and the positions of plural respective points are calculated and represented visually in registration with the generated visual representation of the derived probability density distributions.

In some embodiments, as also exemplified in FIGS. 10-13, a plurality of the new sample data units are received for different subjects and the positions of plural respective points are calculated and represented visually in registration with the generated visual representation of the derived probability density distributions.

In the example of FIGS. 10-12, points in FIG. 10 corresponding to sample data units from a first subject are indicated by arrow 10 and points corresponding to sample data units from a second subject are indicated by arrow 20. Two points 10 are plotted for the first subject. The circular point corresponds to a sample data unit obtained in 2016. The triangular point corresponds to a sample data unit obtained in 2018. Five points 20 are plotted for the second subject, corresponding to sample data units obtained in 2013, 2014, 2016, 2017 and 2018. Confidence scores for each of the sample data units and each of the four disease-relevant classifications are shown in the table to the right of the graph. FIG. 11 shows radar plots representing sample data units from the first subject. The plot depicted by solid circle points and continuous joining lines represents the sample data unit taken from the first subject in 2016. The plot depicted by open circle points and broken joining lines represents the sample data unit taken from the first subject in 2018. FIG. 12 shows radar plots representing sample data units from the second subject. The plot depicted by solid circle points and continuous joining lines represents the sample data unit taken from the second subject in 2013. The plot depicted by open circle points and broken joining lines represents the sample data unit taken from the second subject in 2018.

In the example of FIGS. 13-15, points in FIG. 13 corresponding to sample data units from a third subject are indicated by arrows 31 and 32 and points corresponding to sample data units from a fourth subject are indicated by arrows 41 and 42. The circular point 31 for the third subject corresponds to a sample data unit obtained in 2014. The triangular point 32 for the fourth subject corresponds to a sample data unit obtained in 2018. The circular point 41 for the fourth subject corresponds to a sample data unit obtained in 2015. The triangular point 42 for the fourth subject corresponds to a sample data unit obtained in 2019. Confidence scores for each of the sample data units and each of the four disease-relevant classifications are shown in the table to the right of the graph. FIG. 14 shows radar plots representing sample data units from the third subject. The plot depicted by solid circle points and continuous joining lines represents the sample data unit taken from the third subject in 2014. The plot depicted by open circle points and broken joining lines represents the sample data unit taken from the third subject in 2018. FIG. 15 shows radar plots representing sample data units from the fourth subject. The plot depicted by solid circle points and continuous joining lines represents the sample data unit taken from the fourth subject in 2015. The plot depicted by open circle points and broken joining lines represents the sample data unit taken from the fourth subject in 2019. The examples of FIGS. 10-15 illustrate advantages of the methodology. FIGS. 10-12 depict data from subjects with evidence of a stable disease: a first subject with ET for whom two samples had been taken at an interval of two years, and a second subject with PV who had five bone marrow biopsies performed over six years. In both cases the sequential samples closely aggregated on the plot of FIG. 10, indicating relative stability in their megakaryocytic features. FIGS. 13-15 depict the results of analysis of serial samples from two MPN subjects (the third subject being ET and the fourth subject being PV) who progressed to post-ET and post-PV myelofibrosis, demonstrated by a marked shift in the megakaryocytic classification on the plot of FIG. 13, which was consistent with histological findings for those subjects.

It is noted that the features presented on the radar plots of FIGS. 11-12 and 14-15 do not map consistently onto changes in sample location on the respective PCA plots in FIGS. 10 and 13. A unit change on one radar plot axis may result in a greater/smaller change on the PCA plot. This reflects the different relative weights of each feature and the dimensionality reduction when compressed onto the PCA.

In some arrangements, as exemplified in FIG. 17, the method further comprises performing a cluster analysis to identify a plurality of clusters of the points in the reduced dimension parameter space. Any of a wide variety of known clustering algorithms may be used. An output of the clustering analysis is used to augment the generated visual representation. The generated visual representation may be augmented for example to comprise cluster-boundary indicators 110. The cluster-boundary indicators 110 show the locations of the identified clusters. The cluster-boundary indicators 110 may, for example, comprise closed loops that surround all or a predetermined proportion of points in each identified cluster.

In some arrangements, the generated visual representation comprises a higher-dimensional sample representation 112 for one or more representative sample data units.

Each higher-dimensional sample representation 112 depicts three or more dimensions of a respective sample data unit. Optionally, all of the N-dimensions of the N-dimensional set of values are represented.

In some arrangements, as exemplified in FIG. 17, each higher-dimensional sample representation 112 is visually associated with a respective one of the identified clusters. The higher-dimensional sample representation may, for example, be positioned directly adjacent to a respective cluster-boundary indicator 110 or within a respective cluster-boundary indicator 110. The higher-dimensional sample representation 112 may represent an average over sample data units located in the respective cluster (e.g., an average of each dimension, or feature value, of the N-dimensional sets of values representing the sample data units in the cluster). In the example shown, each higher-dimensional sample representation 112 comprises a radar plot representing an average of feature values in the cluster.

As can be appreciated from FIG. 17, the augmented visual representation allows efficient interpretation of, for example: 1) different subgroups in the disease space and what combination of features are dominant in each subgroup; and 2) changes in the features in the direction of disease progression. The approach highlights the benefit of being able to map between the compressed (e.g. two-dimensional or three-dimensional) representation of sample data units achieved by the dimensionality reduction (which facilitates comparison between cohorts and/or species) and the original higher-dimensional representations that provide more granular information about individual samples, such as morphological characteristics of objects such as cells in an image or information about a topological distribution of such objects in the image.

Any of the methods for generating a visual representation disclosed herein may be performed by a system 100 for generating a visual representation, as depicted schematically in FIG. 16. The system 100 may comprise an imaging device 104. The imaging device 104 is configured to capture an image of a biological sample 106. The system 100 further comprises a data processing system 102 (e.g. a computer). The data processing system 102 is configured to process a sample data unit comprising information derived from the image of the biological sample. The processing comprises obtaining an N-dimensional set of values representing morphological and/or topological characteristics of objects in the image of the biological sample. The processing comprises using a dimensionality reduction algorithm to represent each sample data unit as a respective point in a reduced dimension parameter space. The processing further comprises generating a visual representation of the sample data unit in the reduced dimension parameter space together with a visual representation of probability density distributions for each of plural disease-relevant classifications in the reduced dimension parameter space. In some embodiments, the data processing system 102 comprises a display and the visual representation is generated on the display. In other embodiments, the data processing system 102 generates data representing the visual representation and the data is sent to another device capable of displaying the visual representation using the data.

Further information about the detailed example and other additional details are given below.

### DETAILED EXAMPLE

### Materials and Methods

### Clinical Samples

Sample data units were derived from BMT samples obtained from the archive of OUH NHS Foundation Trust. All specimens were of sufficient technical quality (staining and section thickness) for use in conventional histological reporting and contained at least five intact intertrabecular spaces. Samples were fixed in 10% neutral buffered formalin for 24 hours prior to decalcification in 10% EDTA for 48 hours. Whole slide scanned images (Hamamatsu NanoZoomer 2.0HT/40×/NDPI files) were prepared from 4 μm H&E stained sections cut from formalin-fixed paraffin-embedded (FFPE) blocks. The data set comprised 131 samples (45 ET, 18 PV, 25 MF and 43 reactive/non-neoplastic) with “reactive” samples identified as patients in whom there was no evidence of bone marrow malignancy and no evidence of an underlying myeloid disorder. ET, PV and MF (primary [PMF] or secondary [SMF]) cases represent patients in whom this was either an established or new diagnosis, satisfying the diagnostic criteria of the latest WHO classification (2016), and were designated following review by a myeloid multidisciplinary meeting (MDM). BMTs were identified from the laboratory reporting system or MDM records. This work was conducted as part of the INForMeD study (INvestigating the genetic and cellular basis of sporadic and Familial Myeloid Disorders; IRAS ID: 199833; REC reference: 16/LO/1376; PI: Prof A J Mead).

### Automated Identification of Megakaryocytes

Accurate detection and delineation of megakaryocytes was fully automated using deep-learning based approaches. The detection task comprised predicting the locations of megakaryocytes on a sample using a deep neural network called Single Shot Multibox Detector (Liu W, Anguelov D, Erhan D, et al. Single shot multibox detector. European conference on computer vision: Springer, Cham.; 2016: pp. 21-27). This method defines a default set of bounding boxes over different aspect ratios and scales. To find the megakaryocytes, the network generated a score for each default box to indicate the likelihood that it contained a megakaryocyte and a score for the recommended offset for each default box that more closely matches the identified megakaryocyte. The validity of each predicted bounding box was confirmed by at least one haematopathologist.

Following detection, image segmentation was used to partition the images into different regions to locate the boundaries of objects of interest; in this case megakaryocyte cells. This segmentation task was performed using a method called U-Net which delineates the boundaries of megakaryocytes, segmenting the cell area of interest from the background microenvironment (Ronneberger O, Fischer P, Brox T. Convolutional networks for biomedical image segmentation. International Conference on Medical Image Computing and Computer-Assisted Intervention: Springer, Cham.; 2015: pp. 234-241).

### Megakaryocyte Library

From an initial training set of 2,427 manually annotated megakaryocytes taken from 14 reactive and 6 ET samples, a total of 62,479 delineated megakaryocytes was curated from the whole data set. This library was used for the identification of a set of discrete cytomorphological subtypes. To ensure accurate capture of cytomorphological information, a significant portion (60% [37,284]) of the total megakaryocytes in the library were reviewed by a haematopathologist. This was achieved using an iterative approach in which custom-built computer assisted annotation tools highlighted individual cells within a bounding box and prompted either acceptance or rejection of the enclosed feature as a megakaryocyte.

### Identification of Cytomorphological Subtypes

The inventors learned feature representation, a numerical vector encoding the megakaryocyte cytomorphology, using a type of neural network (autoencoder) that can be trained in an unsupervised manner to learn efficient data encodings. To train the autoencoder, validated megakaryocytes were used from 43 reactive and 30 ET slides. Autoencoder training on reactive and ET samples could be generalising to other subtypes. Validation involved review by haematopathologists who indicated that a detected megakaryocyte was either a true positive detection, an unclear result or a false positive.

Following feature extraction, a clustering analysis was performed to group cytomorphologically similar megakaryocytes. A self-organizing map (SOM) (see Kohonen T. The self-organizing map. IEEE; 1990:1464-1480) was trained on the learned latent representation vectors from reactive (n=43 cases, 7336 cells), ET (n=45 cases, 17535 cells) and PV (n=18 cases, 12413 cells) samples to produce a 10-by-10 grid with 100 megakaryocyte subtypes or groups (as shown in FIG. 2). To further reduce the subtypes, the inventors applied Markov clustering on the self-organising groups with the graph structure determined by the grid configuration (Van Dongen S M. Graph clustering by flow simulation Centre for Mathematics and Computer Science (CWI). Vol. PhD: Amsterdam; 2000). The Markov clustering result that maximises the modularity, i.e. how well nodes on the graph structure are separated into modules (clusters), was used. This resulted in nine distinct cytomorphological subtypes to which each megakaryocyte was assigned (FIG. 2).

### Spatial Distribution Analysis

The spatial density of megakaryocytes was estimated using kernel density with a linear kernel. A single kernel's bandwidth was estimated from reactive (n=43), ET (n=45), and PV (n=18) samples by Silverman's rule-of-thumb (Silverman B W. Density estimation for statistics and data analysis. New York: Routledge; 2018). Clusters of megakaryocytes consist of at least two or more megakaryocytes that are physically touching. The inventors constructed a graph of megakaryocytes where edges link between touching cells and used a Markov clustering algorithm to determine dense clusters.

### Results

### Assisted Megakaryocyte Library Construction

The inventors employed a human-in-the-loop methodology to efficiently build a large library of annotated megakaryocytes (62,729 cells). A web-based annotation tool was used for megakaryocyte identification. The identification tool detected candidate megakaryocytes for which the delineation tool suggested segmentation between the boundaries of the cell cytoplasm and adjacent/background structures. To ensure accuracy and quality, these predicted results were reviewed and edited by specialist haematopathologists and fed into the AI models for further training to iteratively improve the model performance. The tools achieved high levels of accuracy for identification (mean average precision [mAP]=0.86) and delineation (intersection over union [IoU]=0.93) within four training iterations. The time spent on annotating megakaryocytes with the assisted AI tools was significantly less than manual annotation (median detection time: 1.86 s vs. 12.29 s, p=2.2e-16; median segmentation time: 5.41 s vs. 8.62 s, p=3.2e-11).

Megakaryocyte Cytomorphology & Topography and their Diagnostic Associations

An autoencoder neural network was used to identify a feature set that best captures the megakaryocyte cytomorphology. A total of nine cytomorphological subtypes were identified through clustering analysis performed on these learnt features. Each of the nine identified subtypes have distinct, readily appreciated cellular characteristics (FIG. 2). For example, subtypes 8 and 9 are large cells with an atypical, polylobated nucleus. By contrast, cells of subtypes 2 and 3 are small with a high nuclear-cytoplasmic ratio. Several megakaryocyte subtypes are not easily distinguished by haematopathologists, emphasizing the benefits of automated over conventional subjective assessment.

As expected, when compared to reactive samples, the MPN BMTs contained significantly more megakaryocytes, with greater average cell size and heterogeneity in cytological features. MPN megakaryocytes were also significantly more clustered (defined as two or more cells in direct contact) when compared to reactive samples, as determined by the proportion of megakaryocytes within clusters, their density and relative cluster size.

### Morpho-Molecular Association Analysis

The inventors explored whether there was any association between megakaryocyte cytological subtypes and underlying driver mutation status, including TN cases and those carrying the two most common driver mutations (JAK2V617F and CALR). Statistically significant associations were observed for eight of the nine megakaryocyte subtypes. Amongst the associations seen, megakaryocyte subtypes 1 and 4 were significantly under-represented in CALR mutation-bearing samples when compared to both TN and JAK2 mutated cases, while subtype 7 was significantly increased in TN cases compared to JAK2- and CALR-mutated samples.

Some embodiments of the disclosure are defined in the following numbered clauses.
1. A computer-implemented method of generating a visual representation of variation of a disease-relevant classification over a parameter space representing biological samples from human subjects, the method comprising:
receiving training data comprising, for each of a plurality of human subjects, at least one sample data unit comprising information about a biological sample taken from the subject, the training data also comprising a disease-relevant classification of the subject when the biological sample was taken, the information about the biological sample being represented in each sample data unit by an N-dimensional set of values, where N>2;
using a dimensionality reduction algorithm to represent each sample data unit as a respective point in a two-dimensional parameter space;
processing the resulting distributions of points for each of a plurality of disease-relevant classifications to derive a probability density distribution for each of the disease-relevant classifications in the two-dimensional parameter space; and
generating a visual representation of each of the derived probability density distributions in the two-dimensional parameter space, thereby providing a visual representation of disease-relevant classification variation over the parameter space.
   2. The method of clause 1, wherein the dimensionality reduction algorithm uses principle component analysis.
   3. The method of clause 1 or 2, wherein the processing to derive the probability density distributions is performed using kernel density estimation.
   4. The method of any preceding numbered clause, wherein at least a subset of the dimensions of the N-dimensional set of values comprises parameters representing morphological characteristics of objects in an image of the biological sample.
   5. The method of clause 4, wherein at least a subset of the dimensions of the N-dimensional set of values comprises parameters representing a topological distribution of the objects.
   6. The method of clause 4 or 5, further comprising performing feature extraction using machine learning to define at least a subset of the dimensions of the N-dimensional set of values.
   7. The method of any of clauses 4-6, wherein at least a subset of the objects are human cells.
   8. The method of clause 7, wherein at least a subset of the cells are megakaryocytes.
   9. The method of clause 8, wherein the disease-relevant classifications include a classification associated with at least one myeloproliferative neoplasm.
   10. The method of clause 9, wherein the disease-relevant classifications include a classification associated with each of two or more Philadelphia-negative myeloproliferative neoplasms, preferably including one or more of thrombocythaemia, ET, polycythaemia vera, PV, and myelofibrosis, MF.
   11. The method of clause 9 or 10, wherein the disease-relevant classifications include a classification associated with reactive mimics.
   12. The method of any preceding numbered clause, further comprising:
receiving a new sample data unit from a subject to be assessed; and
calculating the position of a point in the two-dimensional parameter space representing the received new sample data unit.
   13. The method of clause 12, wherein the calculation of the position comprises using the dimensionality reduction algorithm to reduce the dimensions of the new sample data unit.
   14. The method of clause 12 or 13, further comprising determining a disease-relevant classification of the new sample data unit, and a quantitative measure of confidence in the determined classification, using the derived probability density distributions and the calculated position of the point in the two-dimensional parameter space representing the received new sample data unit.
   15. The method of any of clauses 12-14, wherein a plurality of the new sample data units are received for the same subject at different times and the positions of plural respective points are calculated and represented visually in registration with the generated visual representation of the derived probability density distributions.
   16. The method of any of clauses 12-15, wherein a plurality of the new sample data units are received for different subjects and the positions of plural respective points are calculated and represented visually in registration with the generated visual representation of the derived probability density distributions.
   17. The method of any preceding numbered clause, wherein the generated visual representation of the derived probability density distributions comprises plots representing one or more contours of equal confidence.
   18. The method of any preceding numbered clause, wherein the generated visual representation is displayed on a display device or output as data suitable for causing a display to display the generated visual representation.
   19. A computer program comprising instructions which, when the program is executed by a computer, cause the computer to carry out the method of any of clauses 1-18.
   20. A computer-readable medium comprising instructions which, when executed by a computer, cause the computer to carry out the method of any of clauses 1-18.
   21. A system for generating a visual representation of variation of a disease-relevant classification over a parameter space representing biological samples from human subjects, comprising:
an imaging device configured to capture an image of a biological sample; and
a data processing system configured to process a sample data unit comprising information derived from the image of the biological sample, wherein the processing comprises:
obtaining an N-dimensional set of values representing morphological and/or topological characteristics of objects in the image of the biological sample;
using a dimensionality reduction algorithm to represent each sample data unit as a respective point in a two-dimensional parameter space; and
generating a visual representation of the sample data unit in the two-dimensional parameter space together with a visual representation of probability density distributions for each of plural disease-relevant classifications in the two-dimensional parameter space.

1. A computer-implemented method of generating a visual representation of variation of a disease-relevant classification over a parameter space representing biological samples from human and/or animal subjects, the method comprising:
receiving training data comprising, for each of a plurality of human and/or animal subjects, at least one sample data unit comprising information about a biological sample taken from the subject, the training data also comprising a disease-relevant classification of the subject when the biological sample was taken, the information about the biological sample being represented in each sample data unit by an N-dimensional set of values, where N>2;
using a dimensionality reduction algorithm to represent each sample data unit as a respective point in a reduced dimension parameter space having fewer than N dimensions;
processing the resulting distributions of points for each of a plurality of disease-relevant classifications to derive a probability density distribution for each of the disease-relevant classifications in the reduced dimension parameter space; and
generating a visual representation of each of the derived probability density distributions in the reduced dimension parameter space, thereby providing a visual representation of disease-relevant classification variation over the parameter space.

2. The method of claim 1, wherein the dimensionality reduction algorithm uses principle component analysis.

3. The method of claim 1, wherein the processing to derive the probability density distributions is performed using kernel density estimation.

4. The method of claim 1, wherein at least a subset of the dimensions of the N-dimensional set of values comprises parameters representing morphological characteristics of objects in an image of the biological sample.

5. The method of claim 4, wherein at least a subset of the dimensions of the N-dimensional set of values comprises parameters representing a topological distribution of the objects.

6. The method of claim 4, further comprising performing feature extraction using machine learning to define at least a subset of the dimensions of the N-dimensional set of values.

7. The method of claim 4, wherein at least a subset of the objects are human and/or animal cells.

8. The method of claim 7, wherein at least a subset of the cells are megakaryocytes.

9. The method of claim 8, wherein the disease-relevant classifications include a classification associated with at least one myeloproliferative neoplasm.

10. The method of claim 9, wherein the disease-relevant classifications include a classification associated with each of two or more Philadelphia-negative myeloproliferative neoplasms, preferably including one or more of thrombocythaemia, ET, polycythaemia vera, PV, and myelofibrosis, MF.

11. The method of claim 9, wherein the disease-relevant classifications include a classification associated with reactive mimics.

12. The method of claim 1, further comprising:
receiving a new sample data unit from a subject to be assessed; and
calculating the position of a point in the reduced dimension parameter space representing the received new sample data unit.

13. The method of claim 12, wherein the calculation of the position comprises using the dimensionality reduction algorithm to reduce the dimensions of the new sample data unit.

14. The method of claim 12, further comprising determining a disease-relevant classification of the new sample data unit, and a quantitative measure of confidence in the determined classification, using the derived probability density distributions and the calculated position of the point in the reduced dimension parameter space representing the received new sample data unit.

15. The method of claim 12, wherein a plurality of the new sample data units are received for the same subject at different times and the positions of plural respective points are calculated and represented visually in registration with the generated visual representation of the derived probability density distributions.

16. The method of claim 12, wherein a plurality of the new sample data units are received for different subjects and the positions of plural respective points are calculated and represented visually in registration with the generated visual representation of the derived probability density distributions.

17. The method of claim 1, wherein the generated visual representation of the derived probability density distributions comprises plots representing one or more contours of equal confidence.

18. The method of claim 1, wherein the generated visual representation is displayed on a display device or output as data suitable for causing a display to display the generated visual representation.

19. The method of claim 1, further comprising performing a cluster analysis to identify a plurality of clusters of points representing the sample data units.

20. The method of claim 19, wherein the generated visual representation comprises cluster-boundary indicators representing locations of the identified clusters in the reduced dimension parameter space.

21. The method of claim 19, wherein the generated visual representation comprises a higher-dimensional sample representation for each of one or more representative sample data units, each higher-dimensional sample representation having more dimensions than the reduced dimension parameter space.

22. The method of claim 21, wherein each higher-dimensional sample representation is visually associated with a respective one of the identified clusters and the representative sample data unit represents one or more sample data units located in the cluster, optionally representing an average over sample data units located in the cluster.

23. The method of claim 1, wherein the reduced dimension parameter space is a two-dimensional parameter space or a three-dimensional parameter space.

24. A computer program comprising instructions which, when the program is executed by a computer, cause the computer to carry out the method of claim 1.

25. A computer-readable medium comprising instructions which, when executed by a computer, cause the computer to carry out the method of claim 1.

26. A system for generating a visual representation of variation of a disease-relevant classification over a parameter space representing biological samples from human and/or animal subjects, comprising:
an imaging device configured to capture an image of a biological sample; and
a data processing system configured to process a sample data unit comprising information derived from the image of the biological sample, wherein the processing comprises:
obtaining an N-dimensional set of values representing morphological and/or topological characteristics of objects in the image of the biological sample;
using a dimensionality reduction algorithm to represent each sample data unit as a respective point in a reduced dimension parameter space; and
generating a visual representation of the sample data unit in the reduced dimension parameter space together with a visual representation of probability density distributions for each of plural disease-relevant classifications in the reduced dimension parameter space.
